SEQUENCE LISTING (5 sequences)

SEQ ID NO: 1 - protein, 124 aa, Bos taurus
KETAAAKFERQHMDSSTSAASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 2 - protein, 124 aa, Artificial Sequence
  mutant peptide
KSTAAAKFSRQHMDSSTSAASSSNYCNQMMKSRNLTKDRCKPVNTFVHSSLADVQAVCSQKNVACKNGQTNCYQSYSTMSITDCRSTGSSKYPNCAYKTTQANKHIIVACSGNPYVPVHFDASV

SEQ ID NO: 3 - protein, 124 aa, Artificial Sequence
  mutant peptide
KSTAAAKFSRQHMSSSTSAASSSNYCNQMMKSRNLTKSRCKPVNTFVHSSLASVQAVCSQKNVACKNGQTNCYQSYSTMSITSCRSTGSSKYPNCAYKTTQANKHIIVACSGNPYVPVHFSASV

SEQ ID NO: 4 - protein, 124 aa, Artificial Sequence
  mutant peptide
KSTAAAKFSRKHMSSSTSAASSSNYCNKMMKSRNLTKSRCKPVNTFVHSSLASVKAVCSQKNVACKNGKTNCYKSYSTMSITSCRSTGSSKYPNCAYKTTKANKHIIVACSGNPYVPVHFSASV

SEQ ID NO: 5 - protein, 124 aa, Artificial Sequence
  mutant peptide
KSTAAAKFSRKHMSSSTSAASSSKYCKKMMKSRNLTKSRCKPVKTFVHSSLASVKAVCSQKKVACKKGKTKCYKSYSTMSITSCRSTGSSKYPKCAYKTTKAKKHIIVACSGKPYVPVHFSASV

(19) United States
(12) Patent Application Publication
(10) Pub. No.: US 2011/0071281 A1
(43) Pub. Date: Mar. 24, 2011

(54) Electrolysis for protein modification

(75) Inventors: Geen-Dong Chang, Taipei City (TW); Der-Yen Lee, Yunlin County (TW)

(73) Assignee: National Taiwan University, Taipei (TW)

(21) Appl. No.: 12/659,844

(22) Filed: Mar. 23, 2010

(30) Foreign Application Priority Data
Sep. 18, 2009 (TW) 98131483

(51) Int. Cl. C07K 1/00; C25B 3/04

(52) U.S. Cl. 530/402; 205/431

(57) ABSTRACT

The present invention relates to a method of protein electrolysis, comprising: (a) providing a protein sample, which contains a salt selected from alkali metal salts or alkaline earth metal salts having a concentration of 20 mM or above; (b) electrolytically reducing said protein sample; (c) obtaining an electroreduced protein sample. The present invention also relates to a protein obtained by the above-mentioned protein electrolysis method, and a use of said method for protein modification.

### FIELD OF THE INVENTION

The present invention relates to a method of protein electrolysis, a protein obtained by the above-mentioned protein electrolysis method, and a use of said method for protein modification.

### BACKGROUND OF THE INVENTION

Since its introduction in 1975, two-dimensional electrophoresis (2-DE) has been widely used for the analysis of protein samples from pure protein preparation to crude tissue extracts. The first dimensional electrophoresis, i.e. isoelectric focusing (IEF), has been improved by using an immobilized pH gradient (IPG) strip. To achieve optimal resolution and visualization, interfering compounds, such as salts, nucleotides, polysaccharides, lipids and particulate materials, need to be removed from the samples prior to IEF. It is generally believed that the presence of high salts would increase conductivity, thereby hindering focusing in IEF. Moreover, rapid transport of water and ions would trigger electroosmosis, which results in protein aggregation and artifacts.

However, electrolytic reactions always accompany the electrophoresis operated at a voltage significantly higher than the redox potential of water, including anodic oxidation generating oxygen gas and protons and cathodic reduction generating hydrogen gas and hydroxide ions. The presence of electrolytes would definitely increase current and therefore increases the rate of electrolysis and power output. Consequently, the pH of the solution at the anode decreases and that at the cathode increases. In addition, bubbles are formed at both electrodes. Accordingly, IEF is usually performed at low voltage for a prolonged time (for instance, at 30 V for 4 hours rather than at 500 V for 1 hour) to reduce the undesired effects caused by high salts. Unfortunately, conventional IEF systems (such as Ettan IPGphore system or Protein IEF system) are closed buffer systems so that the stalled ions would increase the rate of electrolysis.

According to current researches, carbamylation (Righetti et al., 2006; McCarthy et al., 2003), formation of oligomers and β-elimination (i.e. desulfuration) of cysteine resides can only be observed when the samples are inappropriately processed. Proper reduction and alkylation prior to the IEF process can prevent the scrambled disulfide bonds formation and β-elimination of cysteine resides during IEF process. However, protein modifications happened in the high salt IEF process is never described in the previous studies.

### SUMMARY OF THE INVENTION

It is known in the art that electrolytic reduction only occurs at the surface of cathode. Surprisingly, the Inventors find that electrolytic reduction also occurs at a distance from cathode while the electrolysis is performed in the presence of high salts. The present invention uses said high-salt electrolysis to electrolytically reduce a protein and consequently result in the modification of said protein.

Thus, the present invention provides a method of protein electrolysis, comprising the following steps:
(a) providing a protein sample, which contains a salt selected from alkali metal salts or alkaline earth metal salts having a concentration of 20 mM or above;
(b) performing electrolytic reduction of said protein sample;
(c) obtaining an electroreduced protein sample.

In a preferable embodiment of present invention, said electrolytic reduction comprises the following steps: applying said protein sample into a cathodic chamber of an electrolyzer and performing electrolysis.

In a preferable embodiment of present invention, said salt is selected from chloride, bromide, acetate, sulfate or carbonate of an alkali metal or an alkaline earth metal; more preferably, said alkali metal is Li, Na, or K; said alkaline earth metal is Mg or Ca; more preferably, said salt is selected from LiCl, NaCl, NaOAc, Na₂SO₄, CaCl₂, KCl or MgCl₂; even more preferably, from LiCl, NaCl, NaOAc or Na₂SO₄; most preferably, is NaCl.

In a preferable embodiment of present invention, said salt has a concentration of 20 mM to 1000 mM; more preferably, 50 mM to 500 mM; even more preferably, 100 mM to 300 mM; most preferably, 100 mM.

In a preferable embodiment of present invention, said protein sample further comprises an acid; more preferably, said acid is acetic acid, phosphoric acid, citric acid or glycine; even more preferably, is acetic acid.

In a preferable embodiment of present invention, said acid has a concentration of 1˜2%.

In a preferable embodiment of present invention, said step (b) is reducing acid amino residues of said protein sample; more preferably, said step (b) is modifying carboxyl groups of said protein sample into aldehyde groups or alcohol groups.

The present invention also provides a modified protein, which is obtained by the aforesaid method of protein electrolysis. More preferably, said modified protein is a pseudo-protein or a pseudo-peptide.

The present invention also provides a use of the aforesaid method of protein electrolysis for protein modification, which is used for reducing acid amino residues of a protein; more preferably, for modifying carboxyl groups of a protein into aldehyde groups or alcohol groups.

To sum up, the present invention provides a method for protein modification by electrolytic reduction, a modified protein obtained by the method, and a use of said electrolytic reduction method for protein modification. Comparing to traditional chemical methods using reductants, the method of present invention has advantages of low cost, reduced contamination and easily-controllable reaction.

### BRIEF DESCRIPTION OF THE DRAWINGS

FIG. 1 displays the artifacts developed by high salt 2-DE, wherein the first dimensional IEF electrophoresis was performed by using a (A) salt-free, (B) 10 mM NaCl-containing, (C) 20 mM NaCl-containing, (D) 20 mM Na₂SO₄-containing, (E) 20 mM (NH₄)₂SO₄-containing, (F) 20 mM NaCl-containing zebrafish embryo protein sample; wherein, (F) was further performed in-gel dialysis and re-focusing IEF after IEF. The frames in the figures pointed out the anodic and cathodic drift, and the oval-shaped frame in (F) pointed out unexpected artifacts, whereas the oval-shaped frame in (A) pointed out the corresponding location thereof.

FIG. 2 shows the irreversible pI drift of bovine RNase A caused by high salts in 2-DE. (A)˜(B) display the results of denatured IEF, wherein, the first dimensional denatured IEF was performed by using a (A) salt-free, (B) 20 mM NaCl-containing protein sample of bovine RNase A, and (B) was further performed in-gel dialysis and re-focusing IEF after performing the denatured IEF. (C)˜(E) display the results of native IEF, wherein the first dimensional native IEF was performed by using a (C) salt-free, (D) 20 mM NaCl-containing, (E) 20 mM NaCl-containing protein sample of bovine RNase A, and (E) was further performed in-gel dialysis and re-focusing IEF after performing the native IEF.

FIG. 3 demonstrates the RNase A from different time points of a high-salt2-DE, wherein the first dimensional IEF electrophoresis was performed by using a protein sample of RNase A containing 20 mM NaCl at the maximal of 500 V for (A) 0 h, (B) 0.5 h, (C) 1 h, and (D) 1.5 h; wherein all the groups were performed in-gel dialysis and re-focusing IEF after IEF.

FIG. 4 displays effects of electrolysis and electrolytic reduction during IEF. (a) Acidification and anodic drift. (b) Alkalization and cathodic drift. (c) Electrochemically reduced proteins and pI shift. The effects of acidification, anodic drift, alkalization and cathodic drift can be remedied by the in-gel dialysis and refocusing treatment. However, effects of electroreduction and pI shift stay unchanged.

FIG. 5 demonstrates the amino acid sequence comparison between wild-type bovine RNase A (SEQ ID 01) and other variants (SEQ ID 02˜05). D: aspartic acid, E: glutamic acid, S: serine, N: asparagines, Q: glutamine, K: lysine.

FIG. 6 shows the aldehyde group formation of proteins during the high-salt 2-DE, wherein the first dimensional IEF electrophoresis was performed by using a (A) salt-free, (B) 20 mM NaCl-containing, (C) 20 mM NaCl-containing zebrafish embryo protein sample, wherein, (C) was further performed in-gel dialysis and re-focusing IEF after IEF. Streptavidin-peroxidase blot overlay assay was used to detect the reduced protein. ER represented that the RNase A had been electroreduced.

FIG. 7 displays the aldehyde group formation of electroreduced proteins, wherein (A) shows the adducts formed by bPA or bHz and RNase A electroreduced in acetic acid; (B) shows the adducts formed by bPA of various concentrations and ovalbumin electroreduced in acetic acid; (C) shows the adducts formed by electroreduced RNase A or electroreduced ovalbumin and DNPH. The results of above—(A) and (B) were detected by Streptavidin-peroxidase blot overlay assay, whereas, the result of (C) was detected by immunoblotting.

FIG. 8 displays the SDS-PAGE results of un-electroreduced RNase A (RNase A) and electroreduced RNase A (ER—RNase A), wherein the molecular weight of ER-RNase A is higher than that of RNase A.

### DETAIL DESCRIPTION OF THE INVENTION

Except for the definition hereinafter, all the scientific terminologies should be explained as their original means, which is understood by those ordinary skilled in the art. If any argument is caused, the definitions in this specification shall be used as the major explanations.

### DEFINITION

The term “salt” herein is referred as a salt selected from alkali metal salts or alkaline earth metal salts; preferably, chloride, bromide, acetate, sulfate or carbonate thereof; more preferably, any of the aforesaid salts of alkali metal Li, Na, or K; any of the aforesaid salts of alkaline earth metal Mg or Ca; more preferably, a salt selected from LiCl, NaCl, NaOAc, Na₂SO₄, CaCl₂, KCl or MgCl₂; even more preferably, from LiCl, NaCl, NaOAc or Na₂SO₄; most preferably, NaCl. As the protein electrolysis of present invention is achieved by accumulation of free metal ions around cathode and closely related to the electrophoretic mobility of the metal ions, any salt of an alkali metal or an alkaline earth metal is suitable for present invention.

The term “high concentration” herein indicates that the alkali metal salt or alkaline earth metal salt has a concentration of 20 mM or above; preferably, 20 mM to 1000 mM; more preferably, 50 mM to 500 mM; even more preferably, 100 mM to 300 mM; most preferably, 100 mM. Protein modification can be performed on any protein sample by using the alkali metal salt or alkaline earth metal salt having the above-mentioned concentration. The above-mentioned concentration is merely for convenience of regular operation, and those ordinarily skilled in the art can perform protein modification under a salt concentration out of the above-mentioned range by changing other operating conditions accordingly.

The term “acid” herein is referred as any organic or inorganic acid; more preferably, acetic acid, phosphoric acid, citric acid or glycine; even more preferably, acetic acid. The acid is presented to adjust the pH of the electrolytic buffer and to keep the protein in the cathodic chamber. It does not participate in reducing reaction. Therefore, any acid that is able to decrease the pH of electrolytic buffer is suitable for the present invention.

Regarding the two-dimensional electrophoresis in the following examples, the first dimensional isoelectric focusing was performed by using Ettan IPGphor Isoelectric Focusing System (GE Healthcare). First, an IPG gradient strip with pH of 4˜8, 4˜10 or 4˜11 was prepared on GelBond® PAG Film using model 475 Delivery System (Bio-RAD) for gradient making. The length of said strip was 7 cm. Said strip was 4% polyacrylamine gel, wherein the ratio of bisacrylamide to acrylamide was adjusted to 0.045. The concentrations of acrylamido buffer (the 0.2 M stock solution thereof was purchased from Fluka) used for the preparation of said gradient strip were calculated by Doctor pH software (Hoefer). According to the routine condition used in the first dimensional isoelectric focusing, the protein sample was loaded into an IPG strip and the IPG strip was rehydrated for 4˜10 hour. After that, the isoelectric focusing electrophoresis was conducted.

Before the second dimensional electrophoresis, SDS-PAGE, the IPG strips were transferred into a tray, and soaked in a buffer containing 75 mM Tris-HCl (pH 7.8), 0.1% SDS and 0.002% bromophenol blue for 10 minutes, twice. The second dimensional SDS-PAGE electrophoresis was performed by Tris-Tricine SDS-PAGE in accordance with the method disclosed by Schagger etc. (1987). In other words, the SDS-PAGE was performed in 7.5% or 10% polyacrylamide gel while the ratio of bisacrylamide to acrylamide thereof was adjusted to 0.03.

The following examples are just the best exemplary embodiments, and they are not intended to limit the scope of present invention. Those ordinarily skilled in the art can make appropriate changes and amendments according to the disclosure of present invention without departing from the spirits of present invention.

### EXAMPLES

### Example 1

### Proteins Having a Higher pI Value Produced in High-Salt Two-Dimensional Electrophoresis

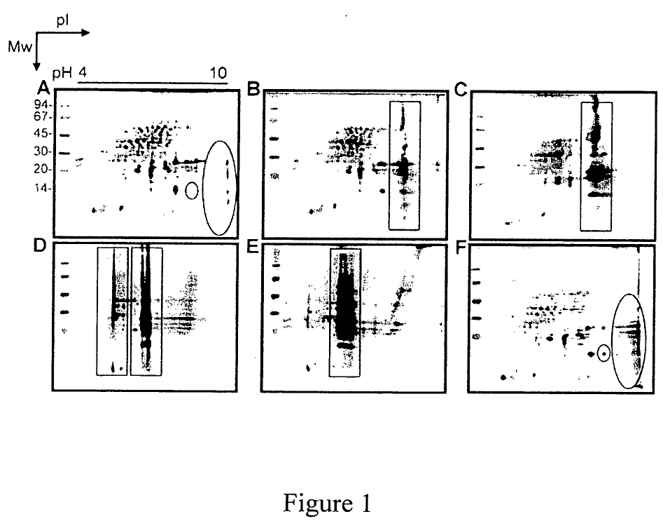

FIG. 1 shows the silver staining result of two-dimensional electrophoresis using the protein sample of Day 1 zebrafish embryos in accordance with the method disclosed by Heukeshoven etc. (1988); wherein The first dimensional IEF electrophoresis was performed for 8,000˜12,000 voltage-hours by using a pH 4˜10 IPG strip previously rehydrated for 4˜10 hours; and the second dimensional SDS-PAGE electrophoresis was performed by using 7.5% polyacrylamine gel.

### Protein Sample Preparation

Day 1 zebrafish embryos were collected and stored at −70° C. For protein sample preparation, one gram of zebrafish embryos were homogenized in 10 ml of cold TE buffer (20 mM Tris-HCL, pH 8.0, 5 mM EDTA) on ice and the homogenate was centrifuged at 27,000×g at 4° C. for 30 minutes. The supernatant was subjected to 90% ammonium sulfate precipitation and the pellet was dissolved in TE buffer. This step was repeated once again and the protein solution was then desalted into the rehydration buffer (8 M urea, 2% CHAPS, 0.5% IPG buffer, pH 3˜10) (GE healthcare) by Millipore centricon YM-10 (Millipore) as a salt-free protein sample.

For high-salt electrophoresis, the 100× stock solution of a salt (such as NaCl, Na₂SO₄ and (NH₄)₂SO₄) was added into said protein sample and said rehydration buffer to achieve the desired final concentration. After rehydrating the IPG strip for 4˜10 hours, the routine IEF (i.e. the first dimensional IEF electrophoresis proceeded for 8,000˜12,000 voltage-hours) can be performed and then the second dimensional electrophoresis is proceeded by using 7.5% polyacrylamide gel. Alternatively, the protein samples in high salts can be subjected to electrophoresis at the maximal voltage of 500 V for 200 voltage-hours as well as in-gel dialysis and re-focusing IEF, and then the second SDS-PAGE electrophoresis is proceeded.

FIG. 1A illustrates the result of the routine IEF and SDS-PAGE electrophoresis by using 20 μg of salt-free protein samples under the aforementioned conditions, and FIG. 1B illustrates the result of the routine IEF and SDS-PAGE electrophoresis by using protein samples with 10 mM NaCl under the aforementioned conditions. In FIG. 1B, obvious protein clumping is shown and it forms a vertical streaking. FIG. 1C illustrates the result of the routine IEF and SDS-PAGE electrophoresis by using protein samples with 20 mM NaCl under the aforementioned conditions. The protein pattern has a poor resolution at sites near the cathode and anode. Protein clumping is shown, and vertical streaking and horizontal streaking are formed.

FIGS. 1D and 1E illustrate the result of the routine IEF and SDS-PAGE electrophoresis by using protein samples with 20 mM Na₂SO₄ and (NH₄)₂SO₄ respectively under the aforementioned conditions. The protein clumping was more pronounced in the presence of high concentration of Na₂SO₄ and (NH₄)₂SO₄.

FIG. 1F shows the result of the following electrophoresis under the aforementioned routine conditions. The protein samples with 20 mM NaCl were subjected to IEF at the maximal voltage of 500 V for 200 voltage-hours. Then the IPG strip was incubated with freshly prepared rehydration buffer (or distilled water) for 5 minutes, twice, to remove excess salts. This step is called in-gel dialysis. After that, the IPG strip was applied to a 7-cm IPG strip holder containing 60 μl of rehydration buffer for performing the second IEF for 8,000˜12,000 voltage-hours. This step is called re-focusing IEF. After re-focusing IEF, the IPG strip was subjected to the above-mentioned second dimensional SDS-PAGE electrophoresis.

According to FIG. 1F, we find that the most artifacts caused by high concentration of salts are not shown in the protein pattern, but extra protein spots appeared at areas close to the cathode. In other words, some of the artifacts in 2-DE caused by high concentration of salts are reversible and can be removed by in-gel dialysis. However, the changes of proteins having higher pI value, which are initially masked because they are unfocused proteins and form protein aggregates, are unmasked by in-gel dialysis and re-focusing IEF.

### Example 2

### Denatured and Native IEF Experiments

In order to closely observe the proteins having high pI value produced in high-salt two-dimensional electrophoresis, a purified bovine RNase A (Sigma) was used for the following experiments.

### 1. Denatured IEF

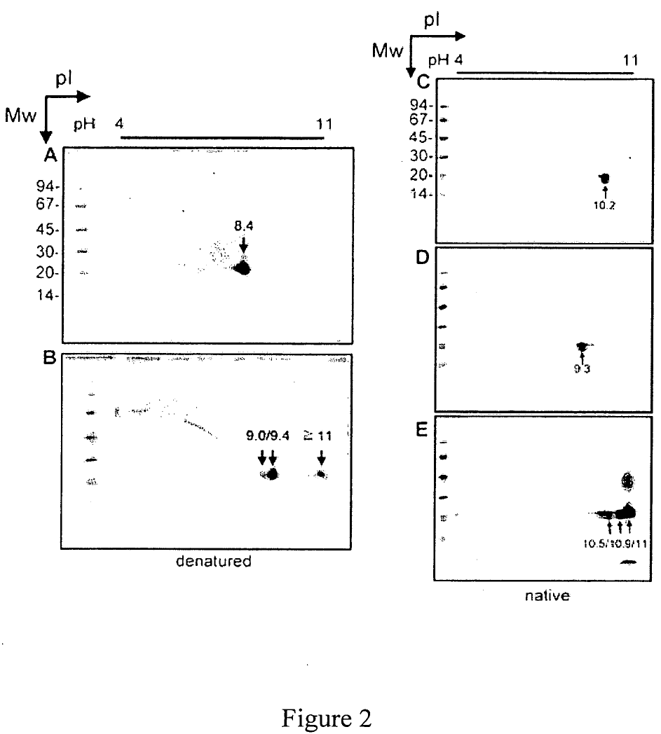

In this experiment, the ribonuclease A (RNase A) protein samples were prepared in rehydration buffer containing 100 mM DTT (2 μg/125 μl). The first dimensional IEF electrophoresis was performed by using a pH 4˜11 IPG strip; the second dimensional SDS-PAGE electrophoresis was performed by using 7.5% polyacrylamide gel; and then silver staining was performed. The rehydration buffer used for in-gel dialysis and re-focusing IEF also contained 100 mM DTT.
A salt-free RNase A protein sample was subjected to the aforementioned routine IEF electrophoresis and SDS-PAGE electrophoresis. The result is shown in FIG. 2A, wherein the denatured RNase A is shown as a protein spot having a pI value of 8.4 and molecular mass of 21 kDa.
Moreover, a RNase A protein sample with 20 mM NaCl was subjected to IEF electrophoresis at the maximal voltage of 500 V for 200 voltage-hours, in-gel dialysis, re-focusing IEF, and then SDS-PAGE electrophoresis. The result is shown in FIG. 2B. The denatured RNase A is shown as three protein spots having a pI value of 9.0, 9.4 and >11.0, respectively, which are all higher than the original pI value.

### 2. Native IEF

Native IEF was applied to eliminate the influence of urea present in rehydration buffer because urea might cause carbamylation of proteins and thereby changing their pI values.
In this experiment, 0.5% IPG buffer was used to replace rehydration buffer for RNase A protein sample preparation (2 μg/125 μl). The first dimensional IEF electrophoresis was performed by using a pH 4˜11 IPG strip; the second dimensional SDS-PAGE electrophoresis was performed by using 7.5% polyacrylamide gel; and then silver staining was performed. The rehydration buffer for in-gel dialysis and re-focusing IEF was replaced by pure water.
A salt-free RNase A protein sample was subjected to the aforementioned routine IEF electrophoresis and SDS-PAGE electrophoresis. The result is shown in FIG. 2C, wherein the native RNase A is shown as a protein spot having a pI value of 10.2 and molecular mass of 21 kDa.
Also, a RNase A protein sample with 20 mM NaCl was subjected to the aforementioned routine IEF electrophoresis and SDS-PAGE electrophoresis. The result is shown in FIG. 2D, wherein the native RNase A is shown as a protein spot having a pI value of 9.3; that is, a pI drift is observed. The pI drift may be caused by electrolysis-generated alkalization.
Last, a RNase A protein sample with 20 mM NaCl was subjected to IEF electrophoresis at the maximal voltage of 500 V for 200 voltage-hours, in-gel dialysis, re-focusing IEF, and then SDS-PAGE electrophoresis. The result is shown in FIG. 2E, wherein the native RNase A is shown as three protein spots having a pI value of 10.5, 10.9 and >11.0, respectively, which are all higher than the original pI value.

In the light of foregoing, proteins having a higher pI value are produced in the absence of protein denaturing agent, such as urea. Therefore, urea does not play a role of protein modification, and the high-salt 2-DE does make an irreversible pI drift of bovine RNase A.

### Example 3

### Irreversible Protein Modification Induced by High Salt 2-DE

In this experiment, 0.5% IPG buffer was used to replace the rehydration buffer for RNase A protein sample preparation (2 μg/125 μl). The first dimensional IEF electrophoresis was performed at the maximal voltage of 500 V for 0, 0.5, 1 or 1.5 hours by using a IPG strip with pH gradient of 4˜11. Also, in-gel dialysis and re-focusing IEF were conducted in accordance with example 1; the second dimensional SDS-PAGE electrophoresis was performed by using 7.5% polyacrylamide gel, and then silver staining was performed.

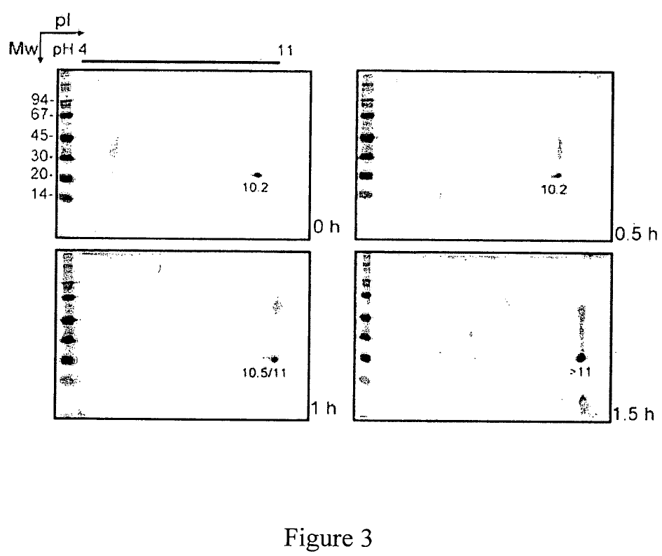

According to the results shown in FIG. 3, the pI value of RNase A protein gradually changed from 10.2 to 10.5 (1 hour) and then to 11.0 (1.5 hours). Apparently, proteins having a high pI value such as RNase A are irreversibly modified during high-salt IEF, and thus obtaining proteins having a higher pI value.

### Example 4

### Protein Alkalization Caused by High-Salt Electrophoresis

Electrolytic reaction plays a key role in the formation of the above-mentioned artifacts including cathodic drift, and that is because electrolysis of water will be enhanced during high salt electrophoresis. Proton accumulation at the anode and hydroxide ion accumulation at the cathode not only cause protonation and deprotonation of proteins at the two electrodes, respectively, but also vanish the pH gradient within the IPG strips near the electrodes. Subsequently, the isoelectric focusing effect is exempted.

In order to determine the influence of pH value on the artifacts, the pH values of the IPG strips were monitored. After IEF, the strips were rinsed by distilled water and then immersed in a solution of phenol red (Merck). Phenol exhibits a color transition from yellow, orange to purple over the pH range 6.8 to 8.4. In the IPG strips used in high salt IEF, the bright yellow band representing acidity and the purple band representing alkalinity moved toward the center of the strips (data not shown).

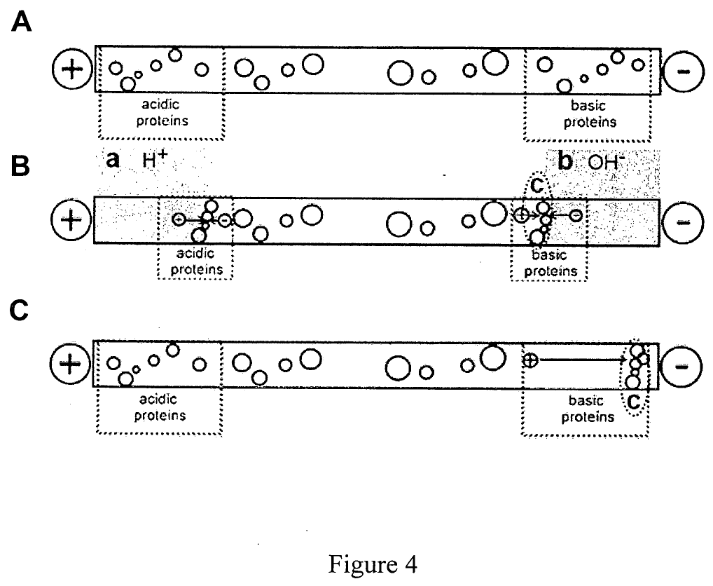

As shown in FIG. 4, a high-salt electrophoresis induces an anodic drift (i.e. protein acidification) and a cathodic drift (i.e. protein alkalization) of proteins. After further examinations with various salts, we find that the level of protein alkalization is the highest in the presence of high concentration of LiCl, NaCl or CaCl₂, and the lowest in the presence of high concentration of (NH₄)₂SO₄, with the descending sequence of LiCl=NaCl>CaCl₂>KCl>MgCl₂>>(NH₄)₂SO₄ (data not shown).

### Example 5

### The Electrolytic Reducing Power of High Salt Electrophoresis

The electrolytic reducing power generated during electrolysis was further examined.

First, IPG strips were rehydrated for 4 hours by 6 mM, 12 mM, 25 mM or 50 mM NaCl or salt-free distilled water. The aforementioned blank strips were subjected to IEF at the maximal voltage of 500 V for 600 or 8,000 voltage-hours. After that, the strips were rinsed in distilled water and stained in 0.5 mg/ml phenol red for 10 sec to reveal the acidification and alkalization of the strips themselves. In addition, IPG strips were rehydrated for 4 hours by 0.5 mg/ml MTT (3-(4,5-dimethylthiazol-2-yl)-2,5-diphenyl-tetrazolium bromide, purchased from Sigma) aqueous solution comprising 6 mM, 12 mM, 25 mM or 50 mM NaCl or the salt-free MTT aqueous solution, and then subjected to IEF at the maximal of 500 V for 600 voltage-hours.

MTT is likely to be reduced to insoluble purple formazan. In the salt-free electrophoresis, there is little MTT being reduced. Relatively, after 600 voltage-hours IEF in the presence of 6 mM or 12 mM NaCl, formazan is produced at sites near cathode; and, after 600 voltage-hours IEF in the presence of 25 and 50 mM NaCl, formazan is localized to 2 to 3 cm away from the cathode (data not shown).

The foregoing results indicate that high concentrations of NaCl interfere with the electrophoresis of MTT, and the cathodic reduction reactions occur away from the cathode. Therefore, localization of MTT at the cathode seems not required for its reduction. Moreover, the extent of MTT reduction is the highest in the presence of high concentration of LiCl, NaCl or CaCl₂, and it is the lowest in the presence of high (NH₄)₂SO₄, with the descending sequence of LiCl=NaCl>CaCl₂>KCl>MgCl₂>>(NH₄)₂SO₄ (data not shown).

Furthermore, application of moist filter paper and anion exchanger paper (size 10×3 mm²) between the electrodes and IPG strip does not affect the MTT reduction. However, the use of cation exchanger paper significantly decreases the MTT reduction at sites near the cathode. This indicates that freely movable cations are necessary for spreading of reducing paper for MTT reduction.

### Example 6

### High-Salt Electrolytic Reduction for Proteins in Acetic Acid Solution

The following experiments were performed by using a home-made horizontal electrolyzer with platinum electrodes. The electrolyzer had a anodic chamber (2.5×3.5×2.0 cm) and a cathodic chamber (2.5×3.5×2.0 cm) that salt-bridged each other with a slice of PVDF membrane (1.8×8.0 cm). Said PVDF membrane had been rinsed sequentially by methanol, water and 1% acetic acid/0.1 M salt in advance. The experiments of this example also can be performed by a commercial electrolyzer.

In the experiments of this example, 5 mg/ml bovine insulin (Sigma) was prepared in 1% acetic acid comprising 0.1 M NaCl or LiCl. The presence of acetic acid can minimize the migration of proteins toward the anodic chamber due to protonation.

3 ml of said bovine insulin sample was loaded into the cathodic chamber while 5 ml of 1% acetic acid comprising 0.1 M NaCl was loaded into the anodic chamber. A small amount of the electrolyzed samples were removed at different time intervals and subjected to SDS-PAGE by using 10% polyacrylamide gel and then staining with GelCode blue dye (purchased from Thermo Scientific) to determine disulfide bond reduction. As results, the reduction of bovine insulin into A and B chains has been detected after electrolyzing for 1.5 hours. After 2 hours electrolysis, and most bovine insulin has been reduced into A and B chains (data not shown). Moreover, the extent of bovine insulin reduction is the highest in the presence of high concentration of LiCl or NaCl, and it is the lowest in the presence of high concentration of (NH₄)₂SO₄, with the descending sequence of LiCl=NaCl>CaCl₂>KCl>MgCl₂>>(NH₄)₂SO₄ (data not shown).

In the light of foregoing, it is clear that alkali and alkaline earth metal ions as electrolytes are able to reduce MTT and protein disulfide bonds, wherein the order of activity among these ions on the reduction of MTT and protein disulfide bonds and the protein alkalization induced by aforesaid electrolysis are identical, and the efficiency of reduction is proportional to ion concentration. Altogether, freely movable metal ions accumulation at sites near the cathode is required for the generation of reducing power.

Moreover, the extent of MTT and protein disulfide bonds reduction is the highest in the presence of high concentration of LiCl or NaCl, and it is the lowest in the presence of high concentration of (NH₄)₂SO₄, with the descending order of LiCl=NaCl>CaCl₂>KCl>MgCl₂>>(NH₄)₂SO₄. The reactivity order is more correlated with the electrophoretic mobility than with the reactivity of these metals with water. Thus, these alkali metal and alkaline earth metal ions are supposed to be reduced to metals at cathode first, and the metals reduce the migrating metal ions, MTT or protein in the gel subsequently. That is a “relay” of reduction. And this is why the reduction occurs not only at cathode, but also at a distance away from the cathode, and as the spreading of reductive power is correlated with the concentration and mobility of cation ions.

### Example 7

### Determination of the Extent of Reduction

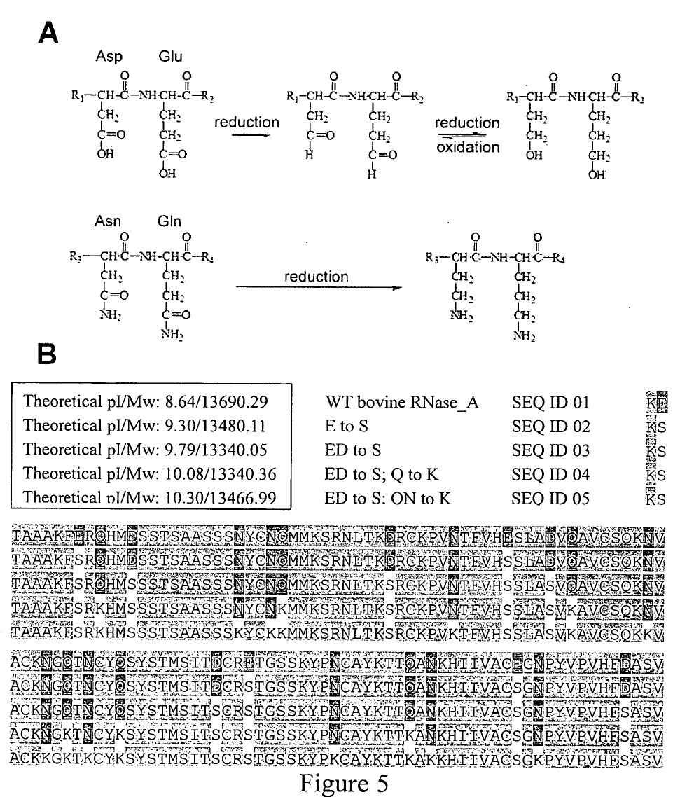

As shown in FIG. 5A, a reductive agent is able to reduce a carboxyl group into an aldehyde or alcohol; and reduce an amide group into a primary amine. Reduction of protein disulfide bonds may not increase the pI value of a given protein. However, side chains of glutamic acid (E), aspartic acid (D), glutamine (Q) and asparagines (N) are prone to cathodic reductions so as to increase the pI values of the given protein.

In order to examine if specific amino acid residues affect the pI of a given protein, four hypothetical bovine RNase A mutants were obtained from the replacement mutation of the protein sequence of wild type RNase A (WT), as shown in FIG. 5B, in which aspartic acid residue (D) and/or glutamic acid residue (E) were replaced by serine residue (S), and asparagines residue (N) and/or glutamine (Q) were replaced by lysine residue (K). The theoretical pI value and molecular weight (Mw) of wild-type and above-mentioned four modified mutants were calculated by pI/Mw tool software on ExPaSy website, wherein the pI/Mw of wild-type bovine RNase A (WT, SEQ ID 01) is 8.64/13690.29; the pI/Mw of mutant a (all E are replaced with S, SEQ ID 02) is 9.30/13480.11; the pI/Mw of mutant b (all E and D are replaced with S, SEQ ID 03) is 9.79/13340.05; the pI/Mw of mutant c (all E and D are replaced with S, and all Q are replaced with K, SEQ ID 04) is 10.08/13340.36; the pI/Mw of mutant d (all E and D are replaced with S, and all Q and N are replaced with K, SEQ ID 05) was 10.30/13466.99.

The results indicate that the reduction of carboxyl groups and amide groups of amino acid residues can convert a given protein to a more basic form. The results are consistent with the experimental data shown in FIGS. 1F, 2 and 3.

### Example 8

### Detection of Aldehyde Group Formation

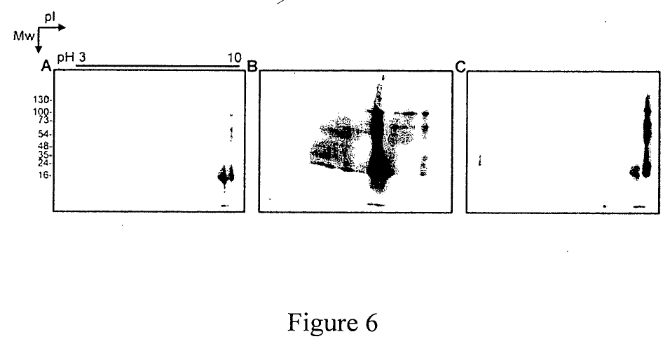

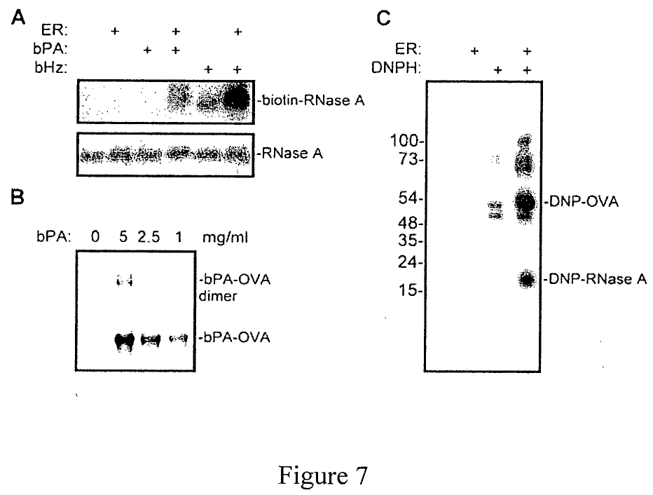

According to the results of aforesaid examples 1˜3, the high-salt 2-DE of present invention is able to reduce a carboxylic acid into the corresponding aldehyde with a carbonyl group (C═O) and even further into the corresponding alcohol. Thus, Schiff base or hydrazone formation, which results from the interaction between aldehyde intermediates and primary amines or hydrazides, can be employed to detect if any aldehyde group of a given protein is formed during a high-salt IEF.
1. The aldehyde group formation of a zebrafish embryo protein sample during 2-DE
   In this example, the zebrafish embryo protein sample according to example 1 further containing 1 mM of EZ-Link® biotin-pentylamine (bPA, purchased from Thermo Scientific) was used, wherein the bPA would conjugate aldehyde groups. An IPG strip with pH gradient of 3˜10 was used in the first dimensional IEF electrophoresis, and 7.5% polyacrylamine gel was used in the second dimensional electrophoresis. After that, 2-DE streptavidin-peroxidase blot overlay assay was performed to detect any bPA adduct of the reduced protein. The rehydration buffer used for in-gel dialysis and re-focusing IEF also contained 1 mM bPA.
   For the streptavidin-peroxidase blot overlay assay, proteins on the gels were transferred at 50 V for 3 hours onto a PVDF membrane in a transfer buffer (50 mM Tris base, 40 mM Glycine, 0.04% SDS and 10% methanol). Then, the PVDF membrane was blocked with 3% BSA in PBS containing 0.05% Tween 20 (PBS-T) for one hour and then washed with PBS-T three times, 5 minutes for each time. After that, the PVDF membrane was probed with 0.01 mg/10 ml horseradish peroxidase-conjugated streptavidin (purchased from Thermo Scientific) in PBS-T containing 0.3% BSA for 30 minutes to let streptavidin and biotin bind to each other. After washing with PBS-T three times (5 minutes for each time), the streptavidin reactive bands on the PVDF membrane were developed by using Immobilon Western ECL (purchase from Millipore).
   For the aforementioned the routine IEF and SDS-PAGE electrophoresis using a salt-free zebrafish embryo protein sample, only a few signals appear near the cathode, as shown in FIG. 6A.
   As shown in FIG. 6B, for the routine IEF electrophoresis using a zebrafish embryo protein sample having 20 mM NaCl, a large amount of aldehyde groups are formed in high-salt concentration environment and the proteins reacted with bPA are clustered at the boundary of cathodic drift because of alkalization.
   Last, an IEF electrophoresis using a zebrafish embryo protein sample having 20 mM NaCl was performed at the maximal voltage of 500 V for 200 voltage-hours, and followed by in-gel dialysis, re-focusing electrophoresis and SDS-PAGE electrophoresis. The result is shown in FIG. 6C. In agreement with above results, it is not necessary for proteins to be electroreduced to locate at the cathode. Furthermore, although in-gel dialysis and re-focusing electrophoresis decrease the extent of aldehyde group formation of proteins, the extent of aldehyde group formation of proteins is still higher than that obtained under routine conditions.
   The modified proteins tend to move toward the higher pH range and form protein oligomers after re-focusing IEF. The results show that high-salt electrophoresis increases the aldehyde groups of proteins, and the proteins are alkalized. In other words, the reduction of carboxyl groups increases the pI value of proteins, and the high-molecular-mass protein oligomers may be resulted from the formation of Schiff base between proteins.
2. The aldehyde group formation of RNase A and ovalbumin in the home-made horizontal electrolyzer of the present invention
   In the light of foregoing, it has been found that the alkali metal and alkaline earth metal ions in high-salt electrophoresis act as an important mediator for electrochemical reduction. Thus, an further electrolytic reduction of proteins was carried out in the home-made horizontal electrolyzer of present invention, and the aldehyde group formation was monitored by addition of bPA, biotin-LC-hydrazide (bHz) or 2,4-dinitrophenyl hydrazide (DNPH).
   First, 5 mg/ml of RNase A protein in 1% acetic acid solution containing 0.1 M NaCl was electrolysed at 100 V for 4 hours. Then, the pH value of the solution was adjusted to 7.0-8.0, and equal volume of 10 mg/ml bPA or 2 mg/ml bHz was added therein. The solution was then incubated at room temperature for 30 minutes for Schiff base formation. Then a SDS-PAGE electrophoresis and a streptavidin-peroxidase blot overlay assay were performed. As shown in FIG. 7A, after high-salt electrophoresis, the RNase A protein reacts with bPA or bHz; wherein the ER in the figure represents the RNase A protein is electroreduced.
   Also, 5 mg/ml of ovalbumin protein (OVA, purchased from Sigma) in 1% acetic acid solution containing 0.1 M NaCl was electrolysed at 100 V for 4 hours. Then, the pH value of the solution was adjusted to 7.0˜8.0, and 10 mg/ml bPA was added therein to a final concentration of 0 mg/ml, 1 mg/ml, 2.5 mg/ml or 5 mg/ml. The solution was then incubated at room temperature for 30 minutes for Schiff base formation. Then SDS-PAGE electrophoresis as well as a streptavidin-peroxidase blot overlay assay is performed. As shown in FIG. 7B, when the concentration of bPA increases, the amount of bPA-ovalbumin adducts increases, too. The bPA-ovalbumin dimer also forms.
   Additionally, an OxyBlot™ protein oxidation detection kit (Chemicon) was used to detect any aldehyde group in the protein side chains which can be derivatized into 2,4-dinitrophenyl hydrazone (DNP-hydrazone) adducts by reaction with DNPH:
   First, 2.5 μl of said electrolyzed RNase A and 2.5 μl of said electrolyzed ovalbumin solution were mixed (1:1) and then 5 μl of 12% SDS as well as 10 μl of 1×DNPH were added and incubated at room temperature for 15 minutes to produce DNP-hydrazone. The reaction was stopped by adding 7.5 μl of neutralizing buffer and 1.5 μl of 2-mercaptoethanol. After that, the sample was subjected to SDS-PAGE and the proteins in the gel were transferred onto a PVDF membrane. After blocking with 1% BSA/PBS-T for 1 hour, the PVDF membrane was added by rabbit anti-DNP antibody (1:150 dilution) in 1% BSA/PBS-T and incubated for 1 hour. After that, the PVDF membrane was washed with PBS-T for 5 minutes, three times, and probed with horseradish peroxidase conjugated goat anti-rabbit IgG antibody (1:200 dilution) in 1% BSA/PBS-T for 1 hour. After washing with PBS-T for 5 minute, three times, the blots were developed by Immobilon Western ECL (Millipore).
   As shown in FIG. 7C, the un-electrolyzed RNase A does not contain any aldehyde groups, whereas, the ovalbumin contains a few aldehyde groups. After electrolyzing in 1% acetic acid containing 0.1 M NaCl at 100 V for 4 hours, the amount of aldehyde group is significantly increased. Thus, the electrolytic reduction of the proteins can be scaled up to a semi-preparative scale for preparing a pseudo-protein whose acidic residues are reduced. The ER in the figure represented that the protein sample has been electroreduecd.
   Furthermore, after quantifying the amount of amino groups of the RNase A, we find that the amine content is decreased after electrolytic reduction. Moreover, the amine content of linear polyacrylamide electrolyzed in water (or 1% acetic acid) containing 0.1 M NaCl at 100 V for 4 hours does not increase either (data not shown). Clearly, amide functional groups cannot be reduced under our electrolysis conditions.

### Example 9

### Molecular Weight Variation Determined by Mass Spectrometric Analysis

To provide additional evidences for reduction of carboxylic acids in the high-salt electrolytic condition of the present invention, a mass spectrometric analysis was accessed to determine the molecular weight of the electroreduced RNase A.

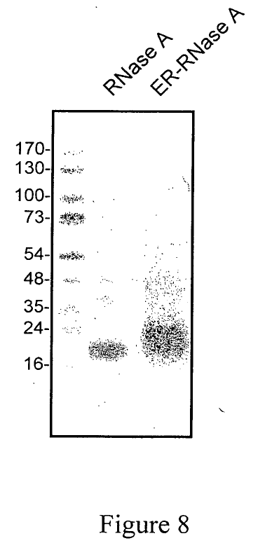

5 μg of un-electroreduced RNase A (RNase A) and electroreduced RNase A (ER-RNase A) were subjected to SDS-PAGE electrophoresis. As shown in FIG. 8A, the molecular weight of ER-RNase A is slightly higher than that of RNase A.

Theoretically, the apparent molecular mass would decrease by 16 or 14 Da if a given carboxylic acid is reduced to an aldehyde or an alcohol, respectively. Alternatively, the apparent molecular mass would decrease by 14 Da if a given amide is reduced to a primary amine. Other possible reactions are reduction of disulfide bonds and formation of Schiff base.

[M+H]⁺/Z of un-electroreduced RNase A peak is 13682.5 while that of electroreduced RNase A peak among a broad range of mass spectra is 13566.6 (data not shown); and the difference of the their molecular weights is 115.9 Da. The molecular weight difference can be resulted from mixed reduction of carboxylic acids, amides, disulfide bonds and formation of Schiff bases (please refer to the following formula),

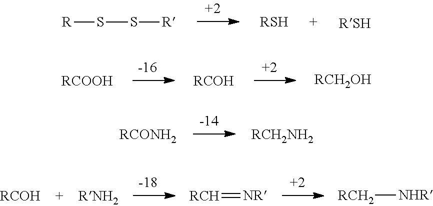

i.e. formation of 2 aldehyde groups (2×16 Da) and 6 amine or alcohol groups (6×14 Da). In addition, higher-molecular-weight forms also appear. The increase in molecular weight cannot be explained by dimerization via Schiff base formation.

Therefore, the high salt electrolysis of present invention can be applied to produce a pseudo-protein or a pseudo-peptide with aldehyde groups, which may be used for conjugation with amine-containing molecules and protein cross-linking, for instance, for conjugation with the amine-containing haptens for immunization.

We claim:

1. A method of protein electrolysis, comprising the following steps:
(a) providing a protein sample, which contains a salt selected from alkali metal salts or alkaline earth metal salts having a concentration of 20 mM or above;
(b) performing electrolytic reduction of said protein sample;
(c) obtaining an electroreduced protein sample.

2. The method according to claim 1, wherein said electrolytic reduction comprises the following steps: applying said protein sample into a cathodic chamber of an electrolyzer and performing electrolysis.

3. The method according to claim 1, wherein said salt is selected from chloride, bromide, acetate, sulfate or carbonate of an alkali metal or an alkaline earth metal.

4. The method according to claim 3, wherein said alkali metal is Li, Na, or K.

5. The method according to claim 3, wherein said alkaline earth metal is Mg or Ca.

6. The method according to claim 1, wherein said salt is selected from LiCl, NaCl, NaOAc, Na₂SO₄, CaCl₂, KCL or MgCl₂.

7. The method according to claim 6, wherein said salt is selected from LiCl, NaCl, NaOAc, or Na₂SO₄.

8. The method according to claim 1, wherein said salt has a concentration of 20 mM to 1000 mM.

9. The method according to claim 1, wherein said protein sample further comprises an acid.

10. The method according to claim 9, wherein said acid is acetic acid, phosphoric acid, citric acid or glycine.

11. The method according to claim 1, wherein said step (b) is reducing acid amino residues of said protein sample.

12. The method according to claim 11, wherein said step (b) is modifying carboxyl groups of said protein sample into aldehyde groups or alcohol groups.

13. A modified protein, which is obtained by the method of claim 1.

14. The modified protein according to claim 13, which is a pseudo-protein or a pseudo-peptide.

15. A use of the method of claim 1 for protein modification, which is used for reducing acid amino residues of a protein.

16. The use according to claim 15, which is used for modifying carboxyl groups of the protein into aldehyde groups or alcohol groups.
